(19) United States
(12) Patent Application Publication
(10) Pub. No.: US 2005/0084518 A1
(43) Pub. Date: Apr. 21, 2005

(54) Health food containing hyaluronic acid and dermatan sulfate

(75) Inventor: Yoshimi Arai, Osaka (JP)

Correspondence Address: Curt Harrington, Suite 250, 6300 State University Drive, Long Beach, CA, 90815, US

(73) Assignee: Medicaraise, Osaka (JP)

(21) Appl. No.: 10/960,233

(22) Filed: Oct. 6, 2004

(30) Foreign Application Priority Data
Oct. 20, 2003 (JP) 2003-360048

(51) Int. Cl. A61K 31/737; A61K 31/728; A61K 47/00

(52) U.S. Cl. 424/439; 514/54

(57) ABSTRACT

It is an object of the present invention to provide a health food containing hyaluronic acid and dermatan sulfate. A health food according to the present invention contains hyaluronic acid and dermatan sulfate as essential components. The health food containing hyaluronic acid and dermatan sulfate according to the present invention makes it possible to take hyaluronic acid and dermatan sulfate together. Further, hyaluronic acid contained in the health food according to the present invention facilitates the absorption of dermatan sulfate in the small intestine and increases the efficiency of absorption of dermatan sulfate.

### BACKGROUND OF THE INVENTION

1. Field of the Invention

The present invention relates to a health food containing hyaluronic acid and dermatan sulfate.

1. Description of the Related Art

Dermatan sulfate, also called chondroitin sulfate B, is one of glycosaminoglycans and has a molecular weight of 20,000 to 400,000. In general, dermatan sulfate is mainly made up of disaccharide repeating units consisting of L-iduronic acid and N-acetylgalactosamine-4-sulfate represented by the following chemical formula 1, but there is a case where some of the repeating units contain sulfated L-iduronic acid or D-glucuronic acid as uronic acid, or contain non-sulfated N-acetylgalactosamine or 4,6-disulfated N-acetylgalactosamine instead of N-acetylgalactosamine-4-sulfate. It is considered that dermatan sulfate is absorbed by the body when orally taken.

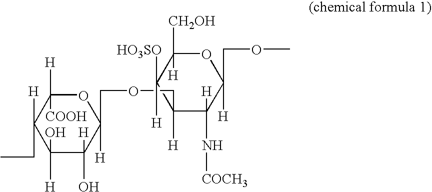

Hyaluronic acid is also one of glycosaminoglycans and has disaccharide repeating units consisting of O-β-D-glucuronosyl(1→3)-N-acetyl-β-D-glucosaminyl(1→4) represented by the following chemical formula 2. Hyaluronic acid is mainly present in the synovial fluid of joints, the vitreous humor of the eye, the umbilical cord, the connective tissues such as upper dermis, and the like of animals. It is considered that hyaluronic acid cannot be absorbed by the body even if it is orally taken because hyaluronic acid has a molecular weight of hundreds of thousands to two millions or more.

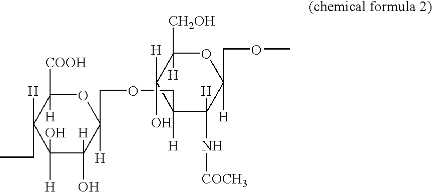

In a living body, dermatan sulfate is linked to hyaluronic acid together with chondroitin sulfate A and chondroitin sulfate C. Since dermatan sulfate, chondroitin sulfate A, chondroitin sulfate C and hyaluronic acid, called glycosaminoglycans, are anionic molecules, when they are linked to each other to form a polymer structure, the resulting polymeric compound can contain a lot of water molecules. Therefore, it is considered that the polymeric compound helps the skin to maintain its moisture. Further, it is pointed out that when orally taken, dermatan sulfate or chondroitin sulfate is got into the body and is then linked to hyaluronic acid present in the body to enhance the effect of maintaining skin's moisture. It is to be noted that some health foods containing hyaluronic acid have been disclosed (see JP-A 2002-360292 and JP-A 09-98739 (1997)), but a health food containing hyaluronic acid and dermatan sulfate together has not yet proposed.

In a case where hyaluronic acid is orally taken, only low-molecular-weight hyaluronic acid (having a molecular weight of 50,000 to 100,000) is absorbed by the body. However, it can be considered that hyaluronic acid orally taken attracts various molecules and plays a role in facilitating the absorption of the molecules in the small intestine. In particular, it can be considered that hyaluronic acid orally taken facilitates the absorption, by the body, of dermatan sulfate, chondroitin sulfate A and chondroitin sulfate C, which are linked to hyaluronic acid in the body.

### SUMMARY OF THE INVENTION

In view of the consideration, the present inventors have produced a health food containing at least hyaluronic acid and dermatan sulfate to investigate the effect thereof As a result, they have found that the health food has excellent effects of maintaining skin's moisture, improving the softness and quality of the skin, and improving health, leading to the completion of the present invention.

It is therefore an object of the present invention to provide a health food containing hyaluronic acid and dermatan sulfate.

The health food containing hyaluronic acid and dermatan sulfate according to the present invention makes it possible to take hyaluronic acid and dermatan sulfate together. Further, hyaluronic acid contained in the health food according to the present invention facilitates the absorption of dermatan sulfate in the small intestine and increases the efficiency of absorption of dermatan sulfate. Furthermore, the health food according to the present invention has the effects of rejuvenating skin, improving the softness of the skin, increasing the moisture retention of the skin, increasing the metabolism of the skin, reducing damage to the skin caused by ultraviolet rays, clearing a muddy complexion, reducing spots on the face, improving appearance of makeup, reducing fine wrinkles, improving skin resilience around eye area, clearing pimples, moisturizing the entire body, improving the color of the nails, curing dry skin, curing chapped lips, improving a ruddy complexion, alleviating stiffness in the shoulders, reducing the risk of a hangover or quickly recovering from a hangover, improving poor circulation, healing wounds faster, strengthening the nails, alleviating backache, reducing memory loss, clearing up blurry vision, alleviating joint pain, improving genital aging, alleviating menstrual pain, softening the skin of the heel, recovering from fatigue, rejuvenating the skin, treating constipation, and reducing hair loss.

### BRIEF DESCRIPTION OF THE DRAWINGS

FIG. 1 is a micrograph of epidermal cells cultured in a culture bottle to which a substance containing hyaluronic acid and dermatan sulfate was added in a concentration of 1.4 mg/mL, which shows a result of Test Example 2;

FIG. 2 is a micrograph of epidermal cells cultured in a culture bottle to which a substance containing hyaluronic acid and dermatan sulfate was added in a concentration of 0.8 mg/mL, which shows a result of Test Example 2;

FIG. 3 is a micrograph of epidermal cells cultured in a culture bottle to which a substance containing hyaluronic acid and dermatan sulfate was added in a concentration of 0.5 mg/mL, which shows a result of Test Example 2;

FIG. 4 is a micrograph of epidermal cells cultured in a culture bottle to which a substance containing hyaluronic acid and dermatan sulfate was added in a concentration of 0.25 mg/mL, which shows a result of Test Example 2;

FIG. 5 is a micrograph of epidermal cells cultured in a culture bottle to which a substance containing hyaluronic acid and dermatan sulfate was added in a concentration of 0.125 mg/mL, which shows a result of Test Example 2;

FIG. 6 is a micrograph of epidermal cells cultured in a culture bottle to which a substance containing hyaluronic acid and dermatan sulfate was added in a concentration of 0.0625 mg/mL, which shows a result of Test Example 2;

FIG. 7 is a micrograph of epidermal cells cultured in a culture bottle to which no substance containing hyaluronic acid and dermatan sulfate was added, which shows a result of Test Example 2;

FIG. 8 is a photograph which shows a cross section of skin exposed to ultraviolet rays;

FIG. 9 is a photograph which shows a cross section of skin exposed to ultraviolet rays; and

FIG. 10 is a photograph which shows a cross section of skin.

### DESCRIPTION OF THE PREFERRED EMBODIMENTS

A health food according to the present invention contains hyaluronic acid and dermatan sulfate as essential components.

Dermatan sulfate, also called chondroitin sulfate B, is one of glycosaminoglycans and has a molecular weight of 20,000 to 400,000. In general, dermatan sulfate is mainly made up of disaccharide repeating units consisting of L-iduronic acid and N-acetylgalactosamine-4-sulfate represented by the following chemical formula 3, but there is a case where some of the repeating units contain sulfated L-iduronic acid or D-glucuronic acid as uronic acid, or contain non-sulfated N-acetylgalactosamine or 4,6-disulfated N-acetylgalactosamine instead of N-acetylgalactosamine-4-sulfate. It is considered that dermatan sulfate is absorbed by the body when orally taken.

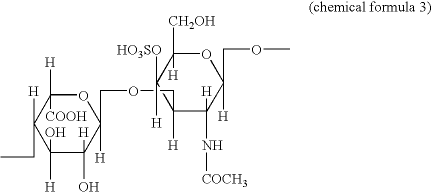

Hyaluronic acid is also one of glycosaminoglycans and has disaccharide repeating units consisting of O-β-D-glucuronosyl(1→3)-N-acetyl-β-D-glucosaminyl(1→4) represented by the following chemical formula 4. Hyaluronic acid is mainly present in the synovial fluid of joints, the vitreous humor of the eye, the umbilical cord, the connective tissues such as upper dermis, and the like of animals. It is considered that hyaluronic acid cannot be absorbed by the body even if it is orally taken because hyaluronic acid has a molecular weight of hundreds of thousands to two millions or more.

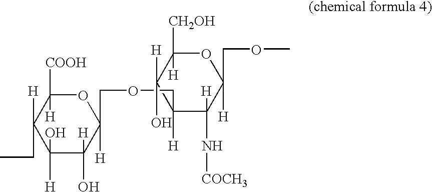

Hyaluronic acid and dermatan sulfate to be used in the present invention may be either synthetic products or semisynthetic products, or may be natural extracts derived from birds, fishes, mammals and the like. In this regard, it is to be noted that a synthetic product refers to one produced by chemical synthesis, and a semisynthetic product refers to one obtained by further carrying out synthesis using a chemical synthetic product or a natural extract. In a case where hyaluronic acid and dermatan sulfate extracted from natural sources are used, extraction sources are not particularly limited, but are preferably mammals to which human beings belong. Among mammals, pigs (genus Sus) are more preferably used as natural sources because it is said that tissue compatibility between pigs and human beings is high. Examples of species of pigs may include Duroc, Berkshire, Hampshire, Landrace, Large Yorkshire (Large White) and Middle Yorkshire (Middle White), and hybrids between two species of them.

The mixing ratio between hyaluronic acid and dermatan sulfate is not limited to any specific value, but the weight ratio between hyaluronic acid and dermatan sulfate to be mixed is preferably 1:0.001 to 200, more preferably 1:0.005 to 100. This is because it can be considered that dermatan sulfate cannot be efficiently absorbed if there is a significant difference between hyaluronic acid and dermatan sulfate in the mixing ratio. Further, there is a possibility that some people have an allergic reaction so that pimples break out on their skin.

The health food containing hyaluronic acid and dermatan sulfate according to the present invention may contain chondroitin sulfate A, chondroitin sulfate C and peptide in addition to the essential components, hyaluronic acid and dermatan sulfate. The addition of chondroitin sulfate A and chondroitin sulfate C makes it possible to synergistically enhance the effects obtained by the health food containing hyaluronic acid and dermatan sulfate according to the present invention, such as the effects of maintaining skin's moisture, improving the softness and quality of the skin, and improving health. Further, addition of peptide makes it possible to synergistically enhance the effect of rejuvenating skin tissue.

Chondroitin sulfate A, also called chondroitin-4-sulfate, is one of glycosaminoglycans, and often refers to chondroitin sulfate having sulfate groups at position 4. Chondroitin sulfate A has a molecular weight of several thousands to several tens of thousands, and the chain length and fine structure thereof vary depending on species of animals, age, and type or region of tissue.

Chondroitin sulfate C, also called chondroitin-6-sulfate, is one of glycosaminoglycans, and often refers to chondroitin sulfate having sulfate groups at position 6. The molecular weight of chondroitin sulfate C is the same as that of chondroitin sulfate B.

Chondroitin sulfate A and/or chondroitin sulfate C to be added to the health food containing hyaluronic acid and dermatan sulfate according to the present invention is not particularly limited, but is preferably extracted from mammals to which human beings belong. Among mammals, pigs (genus Sus) are more preferably used as extraction sources because it is said that tissue compatibility between pigs and human beings is high.

The amount of chondroitin sulfate A and/or chondroitin sulfate C to be added is not limited to any specific value, but the weight ratio between chondroitin sulfate A and/or chondroitin sulfate C and hyaluronic acid to be mixed is preferably about 0.001 to 100:1, more preferably about 0.01 to 50:1, even more preferably about 0.05 to 20:1. This is because it can be considered that chondroitin sulfate A and/or chondroitin sulfate C cannot be efficiently absorbed if there is a significant difference between chondroitin sulfate A and/or chondroitin sulfate C and hyaluronic acid in the mixing ratio. Further, there is a possibility that some people have an allergic reaction so that pimples break out on their skin.

The kind of peptide to be added to the health food containing hyaluronic acid and dermatan sulfate according to the present invention is not particularly limited, but one consisting of 50,000 or less amino acids should be added. Specifically, one consisting of about 5 to 5,000 amino acids is preferable, one consisting of about 5 to 500 amino acids is more preferable, and one consisting of about 5 to 50 amino acids is even more preferable. This is because if peptide consisting of too many amino acids is used, there is a fear that the peptide cannot be absorbed by the body, that is, it can be considered that the smaller the number of linked amino acids is, the easier the body can absorb the peptide.

Such peptide may be extracted from any source, but is preferably extracted from mammals to which human beings belong. Among mammals, pigs are more preferably used as extraction sources because it is said that tissue compatibility between pigs and human beings is high. Alternatively, peptide having a high affinity for human tissue or peptide having a particular effect may be produced by chemical synthesis to add to the health food of the present invention.

The amount of peptide to be added is not limited to any specific value, but the weight ratio between peptide and hyaluronic acid to be mixed is preferably about 0.0001 to 200:1, more preferably about 0.001 to 100:1, even more preferably about 0.01 to 50:1. This is because it can be considered that peptide cannot be efficiently absorbed if there is a significant difference between hyaluronic acid and peptide in the mixing ratio. Further, there is a possibility that some people have an allergic reaction so that pimples break out on their skin.

Hyaluronic acid, dermatan sulfate, chondroitin sulfate A and chondroitin sulfate C to be added to the health food containing hyaluronic acid and dermatan sulfate according to the present invention may be derived from different living things, respectively, or may be synthesized, but they are preferably derived from the same mammal. Among mammals, pigs (genus Sus) are more preferably used as extraction sources because it is said that tissue compatibility between pigs and human beings is high. This is because it can be considered that hyaluronic acid, dermatan sulfate, chondroitin sulfate A and chondroitin sulfate C derived from the same living thing have a high affinity between molecules so that dermatan sulfate, chondroitin sulfate A and chondroitin sulfate C are efficiently absorbed through hyaluronic acid by the body.

The health food containing hyaluronic acid and dermatan sulfate according to the present invention may contain components that are generally used for health foods, in addition to the essential components, hyaluronic acid and dermatan sulfate, and supplemental components, chondroitin sulfate A, chondroitin sulfate C and peptide. Examples of such components to be added may include various kinds of vitamins, collagen, propolis, royal jelly, cellulose, sugar, citric acid, plant extracts, flavoring ingredients, preservatives and the like.

The health food containing hyaluronic acid and dermatan sulfate according to the present invention may have any shape. For example, the health food of the present invention may be formed to be powders, granules, capsules, tablets, liquids or the like. Further, the health food of the present invention may be added to cookies, biscuits, gum, candies, noodles, drinks or the like to produce foods for promoting health.

The ratio of hyaluronic acid, dermatan sulfate and other additives with respect to the entire health food containing hyaluronic acid and dermatan sulfate according to the present invention is not limited to any specific value, but is preferably in the range of about 0.0001 to 90 wt % with respect to the total weight of the health food, more preferably in the range of about 0.001 to 80 wt %, even more preferably in the range of about 0.01 to 70 wt %. In this regard, it is to be noted that the ratio of hyaluronic acid, dermatan sulfate and other additives with respect to the total weight of the health food actually varies depending on the form of the health food to be produced, but preferably lies in the range described above. If the amount of hyaluronic acid, dermatan sulfate and other additives contained in the health food is less than 0.0001 wt %, the effects of the present invention cannot be obtained. On the other hand, even if the amount of hyaluronic acid, dermatan sulfate and other additives contained in the health food exceeds 90 wt %, a higher degree of effectiveness cannot be obtained.

Hereinafter, the effects of the health food containing hyaluronic acid and dermatan sulfate according to the present invention will be described with reference to test examples.

(Test for Evaluating Usefulness of Health Food containing Hyaluronic Acid and Dermatan Sulfate)

An administration test using a health food containing hyaluronic acid and dermatan sulfate was carried out on 40 females in their 40s to 50s for 2 months from Aug. 1 to Oct. 1 in 2003 (that is, from a humid season in summer to a dry season in autumn). In this regard, it is to be noted that the examinees did not take any other health foods and had skin troubles such as “muddy complexion”, “presence of fine wrinkles”, “loss of skin resilience”, “presence of thick layer of dead skin on the heel”, and the like.

The health food used in this administration test was in tablet form, and the examinees took 3 tablets (300 mg per 1 tablet) at bedtime every day. It is to be noted that 300 mg of the tablet contains 5 mg of a hyaluronic acid-containing substance and 5 mg of a dermatan sulfate-containing substance.

### Test Example 1

Evaluation of Moisture content of Skin and Softness of Skin

For the examinees, the moisture content of the skin in the cheek and the moisture content of the skin in an area around the mouth were measured using a skin analyzer (which is manufactured and sold by Tanita Corporation under the product name of Piera). The moisture content of the skin refers to the amount of water contained in the stratum corneum, and is generally measured for checking the degree of moisture retention of the skin. The softness of the skin refers to the degree of softness of the skin, and is generally measured for checking the resilience of the skin.

In this test, data about the examinees, such as sexuality, age, and make-up or no make-up had been already input to a memory in the skin analyzer. Based on the data and information obtained by a tactile sensor attached in the tip of the analyzer, the skin analyzer displayed the graphs of the moisture content of the skin, the softness of the skin and the amount of sebum, and skin age. In this regard, it is to be noted that the amount of sebum was considered to be a factor having no bearing on the effect of the health food of the present invention, and was left out of the results. Each of the moisture content of the skin and the softness of the skin was read from the height of the graph on the display and skin age was directly read from the display to record as measurement values. Measurement was carried out before the examinees started to take the health food containing hyaluronic acid and dermatan sulfate and after a month and 2 months from the beginning of the test.

Evaluation was made by comparing the measurement value before taking of the health food with the measurement value after a month or 2 months from beginning of the test. As for the evaluation results of skin age, the number of people whose skin age became younger than their actual age is shown in Tables 1 and 2 as the “number of people whose measurement values were improved”, the number of people whose skin age became older than their actual age is shown in Tables 1 and 2 as the “number of people whose measurement values became worse”, and the number of people whose skin age was the same as their actual age is shown in Tables 1 and 2 as the “number of people whose measurement values were not changed”. It is to be noted that Table 1 shows the evaluation results as to the cheek, and Table 2 shows the evaluation results as to an area around the mouth.

As for the evaluation results of the moisture content of the skin and the softness of the skin, the number of people whose measurement values were increased is shown in Tables 1 and 2 as the “number of people whose measurement values were improved”, the number of people whose measurement values were decreased is shown in Tables 1 and 2 as the “number of people whose measurement values became worse”, and the number of people whose measurement values were not changed is shown in Tables 1 and 2 as the “number of people whose measurement values were not changed”. As described above, Table 1 shows the evaluation results as to the cheek, and Table 2 shows the evaluation results as to an area around the mouth.

It is to be noted that in a case where the values of the moisture content of the skin and/or the softness of the skin lie in an abnormal level, the skin analyzer used in this test cannot display measurement values. Therefore, there was a case where the moisture content of the skin and/or the softness of the skin could not be measured before taking of the health food so that the measurement values thereof could not be obtained. In a case where the moisture content of the skin and/or the softness of the skin could not be measured before taking of the health food but they could be measured after a month or 2 months from the beginning of the test, it was considered that skin condition was improved.

TABLE 1

| | 1 month after | | | 2 months after | | |
|---|---|---|---|---|---|---|
| | beginning of test | | | beginning of test | | |
| | | | Moisture | | | Moisture |
| | Skin age | Softness | content | Skin age | Softness | content |
| Number of people | 20 | 28 | 32 | 38 | 36 | 39 |
| whose measurement | | | | | | |
| values were improved | | | | | | |
| Number of people | 12 | 5 | 8 | 0 | 0 | 0 |
| whose measurement | | | | | | |
| values became worse | | | | | | |
| Number of people | 8 | 7 | 0 | 2 | 4 | 1 |
| whose measurement | | | | | | |
| values were not | | | | | | |
| changed | | | | | | |

TABLE 2

| | 1 month after | | | 2 months after | | |
|---|---|---|---|---|---|---|
| | beginning of test | | | beginning of test | | |
| | | | Moisture | | | Moisture |
| | Skin age | Softness | content | Skin age | Softness | content |
| Number of people | 26 | 31 | 35 | 37 | 34 | 39 |
| whose measurement | | | | | | |
| values were improved | | | | | | |
| Number of people | 4 | 3 | 2 | 0 | 1 | 0 |
| whose measurement | | | | | | |
| values became worse | | | | | | |
| Number of people | 10 | 6 | 3 | 3 | 5 | 1 |
| whose measurement | | | | | | |
| values were not | | | | | | |
| changed | | | | | | |

As can be seen from the results shown in Tables 1 and 2, in all the measurement items for the cheek, skin age, softness and moisture content after a month from the beginning of the test, people whose measurement values were improved occupied the majority of the examinees. At the time when 2 months had passed from the beginning of the test, the measurement values of 95% of the examinees were improved in skin age, the measurement values of 90% of the examinees were improved in softness, and the measurement values of 97.5% of the examinees were improved in moisture content.

Similarly, in all the measurement items for an area around the mouth, skin age, softness and moisture content after a month from the beginning of the test, people whose measurement values were improved occupied the majority of the examinees. At the time when 2 months had passed from the beginning of the test, the measurement values of 92.5% of the examinees were improved in skin age, the measurement values of 85% of the examinees were improved in softness, and the measurement values of 97.5% of the examinees were improved in moisture content.

As described above, the substance containing hyaluronic acid and dermatan sulfate makes it possible to rejuvenate the skin and to improve the softness of the skin and the moisture content of the skin. Further, it is apparent from the data mentioned above that the substance containing hyaluronic acid and dermatan sulfate functions as an agent for rejuvenating the skin, an agent for softening skin tissue, or an agent for maintaining skin's moisture.

### Test Example 2

Epidermal Cell Culture Experiment using Mouse

About 1 cm² of epidermal tissue of a Kunming species mouse (which was about one day old) was cut out, and cells were separated using protease. A filtered tissue fluid was collected by a sterilized pipet, and the tissue fluid was subjected to centrifugal separation at 1,000 rpm for 10 minutes.

The resulting supernatant was removed, and then MEM medium (containing 20% FES) for precipitate was added to the resulting precipitate. The epidermal cells were observed using 1% placenta fluid. The following experiment was carried out after the growth rate of the epidermal cells reached 98%.

(Experiment)

Culture bottles were prepared, and then 0.2 mL of fluid containing the cells and 10 mL of MEM medium were placed in each of the culture bottles to culture cells at 37° C. in 5% CO₂. A substance containing hyaluronic acid and dermatan sulfate was added to the culture bottles so that the concentration thereof became 1.4 mg/mL, 0.8 mg/mL, 0.5 mg/mL, 0.25 mg/mL, 0.125 mg/mL, 0.0625 mg/mL and 0 mg/mL, respectively, and then cell growth was observed for 7 days. The results are shown in Table 3.

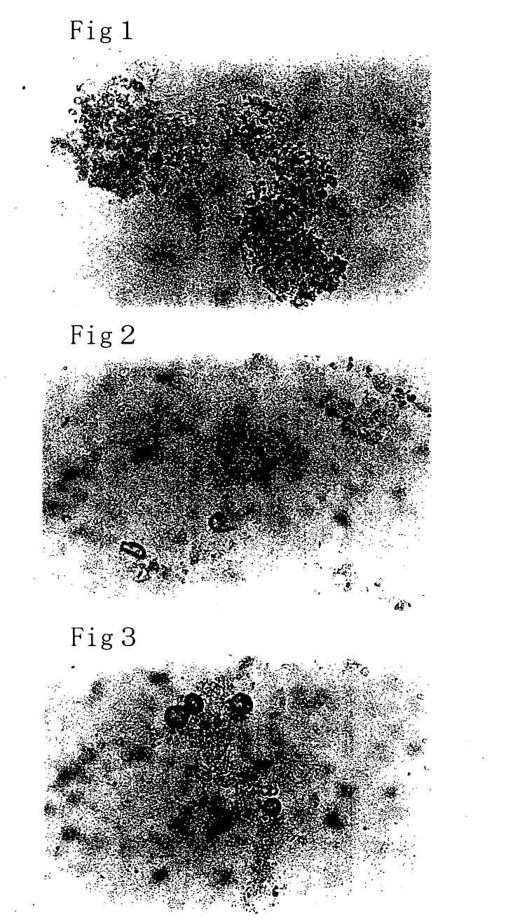

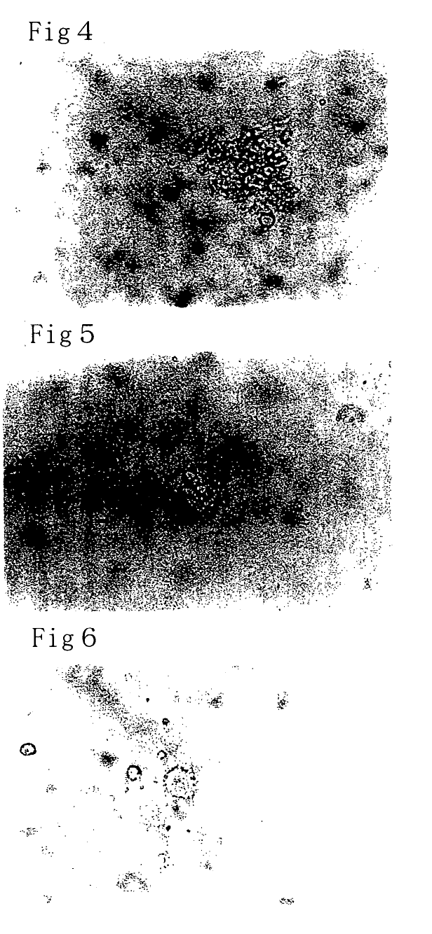

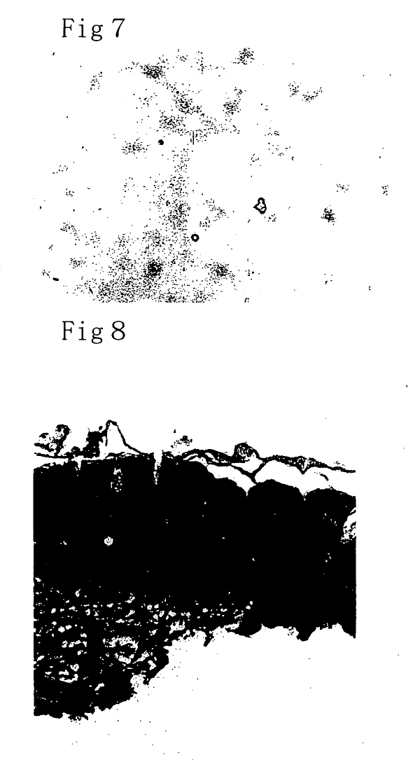

Further, the epidermal cells cultured for 7 days in each of the culture bottles were micrographed. FIG. 1 shows a micrograph of the epidermal cells cultured in the culture bottle to which the substance containing hyaluronic acid and dermatan sulfate was added in a concentration of 1.4 mg/mL, FIG. 2 shows a micrograph of the epidermal cells cultured in the culture bottle to which the substance was added in a concentration of 0.8 mg/mL, FIG. 3 shows a micrograph of the epidermal cells cultured in the culture bottle to which the substance was added in a concentration of 0.5 mg/mL, FIG. 4 shows a micrograph of the epidermal cells cultured in the culture bottle to which the substance was added in a concentration of 0.25 mg/mL, FIG. 5 shows a micrograph of the epidermal cells cultured in the culture bottle to which the substance was added in a concentration of 0.125 mg/mL, FIG. 6 shows a micrograph of the epidermal cells cultured in the culture bottle to which the substance was added in a concentration of 0.0625 mg/mL, and FIG. 7 shows a micrograph of the epidermal cells cultured in the culture bottle to which the substance was added in a concentration of 0 mg/mL.

TABLE 3

| | | Growth rate of epidermal cells (%) | | | | | | |
|---|---|---|---|---|---|---|---|---|
| | | Day 1 | Day 2 | Day 3 | Day 4 | Day 5 | Day 6 | Day 7 |
| Concentration | 1.4 | 8.58 | 17.14 | 25.71 | 34.24 | 42.85 | 51.4 | 59.79 |
| (mg/mL) | 0.8 | 7.17 | 14.29 | 21.38 | 28.56 | 35.5 | 42.84 | 49.98 |
| | 0.5 | 5.71 | 11.41 | 17.14 | 22.9 | 28.55 | 34.26 | 40.02 |
| | 0.25 | 4.24 | 8.56 | 12.83 | 17.13 | 21.4 | 25.73 | 29.76 |
| | 0.125 | 3.28 | 6.54 | 9.84 | 13.15 | 16.46 | 19.68 | 22.96 |
| | 0.0625 | 2.26 | 4.56 | 6.84 | 9.17 | 11.43 | 13.69 | 15.86 |
| | 0 | 0 | 0 | 1.19 | 2.39 | 3.42 | 4.59 | 5.54 |

As can be seen from the results shown in Table 3 and FIGS. 1 to 7, the substance containing hyaluronic acid and dermatan sulfate tends to increase the growth rate of the cells. Further, the higher the concentration of the substance containing hyaluronic acid and dermatan sulfate is, the higher the growth rate of the cells is. From the results, it is apparent that the substance containing hyaluronic acid and dermatan sulfate increases skin's metabolism and that the substance hyaluronic acid and dermatan sulfate functions as an agent for facilitating metabolism.

### Test Example 3

Ultraviolet Rays Irradiation Experiment

4 Kunming species mice (including 2 male mice and 2 female mice) were prepared, and the back of each of the mice was shaved to provide skin to be exposed to ultraviolet rays. The skin was irradiated with 30 W of ultraviolet rays between 7 a.m. and 5 p.m. every day. The 4 mice were divided into 2 pairs each including 1 male mouse and 1 female mouse. To each of the male mouse and the female mouse in one of the pairs, a substance containing hyaluronic acid and dermatan sulfate was administered in an amount of 1,920 mg per kg body weight per day. After a seven-day irradiation with ultraviolet rays, the cross section of the skin exposed to ultraviolet rays was photographed. The results are shown in FIGS. 8 to 10.

FIG. 8 shows a photograph of the cross sections of the skin exposed to ultraviolet rays of the pair of mice to which the substance containing hyaluronic acid and dermatan sulfate was not administered. In this pair of mice, thickening of the epidermis, shedding of the stratum corneum due to the lack of basic substances, loss of power for growing new hair, and drying of the skin were confirmed.

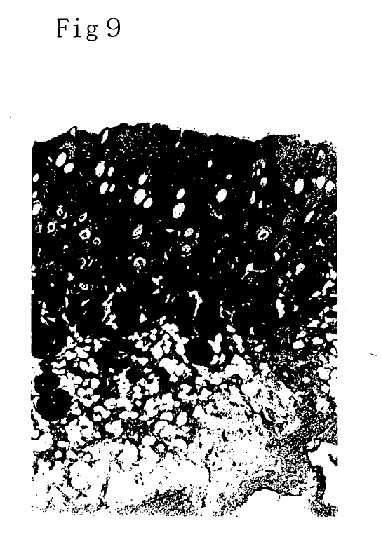

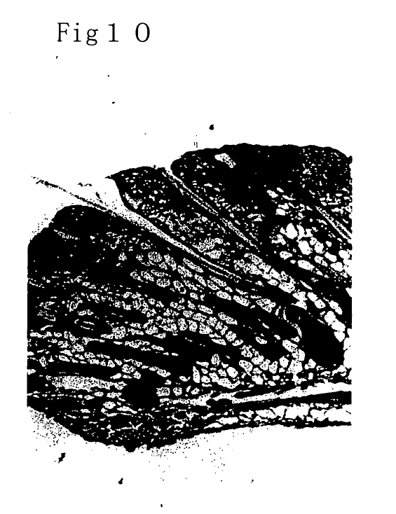

FIG. 9 shows a photograph of the cross sections of the skin exposed to ultraviolet rays of another pair of mice to which the substance containing hyaluronic acid and dermatan sulfate was administered. In this pair of mice to which the substance containing hyaluronic acid and dermatan sulfate was administered, the dermis and subcutaneous tissue were increased, and basic substances were supplied to emerging hair follicles. That is, the skin condition of the mice was close to the condition of the skin that was not irradiated with ultraviolet rays (see FIG. 10).

From the results, it can be considered that the health food containing hyaluronic acid and dermatan sulfate according to the present invention has the effect of reducing damage to the skin caused by ultraviolet rays. Further, it is apparent that substance containing hyaluronic acid and dermatan sulfate functions as an agent for reducing damage to the skin caused by ultraviolet rays.

(Monitoring of Health Food containing Hyaluronic Acid and Dermatan Sulfate)

Monitoring of the health food containing hyaluronic acid and dermatan sulfate according to the present invention was carried out by 20 males and 80 females. The results are shown in Table 4.

TABLE 4

| | Period of use | | | | | | | | | | | | | | |
|---|---|---|---|---|---|---|---|---|---|---|---|---|---|---|---|
| | Day 3 | | Day 6 | | Day 10 | | Day 15 | | Day 20 | | Day 30 | | Total | | Total |
| | Sexuality | | | | | | | | | | | | | | |
| | | | | | | | | | | | | | | | Male |
| | | Fe- | | Fe- | | Fe- | | Fe- | | Fe- | | Fe- | | Fe- | and |
| Effects | Male | male | Male | male | Male | male | Male | male | Male | male | Male | male | Male | male | Female |
| Muddy complexion was | | | | | | 9 | | 10 | 1 | 4 | 2 | 22 | 3 | 45 | 48 |
| cleared | | | | | | | | | | | | | | | |
| Spots on face were reduced | | | | 1 | | 3 | 1 | 6 | 4 | 13 | 3 | 24 | 8 | 47 | 55 |
| Appearance of makeup was | | 37 | | 3 | | 9 | | 12 | | 2 | | 4 | | 67 | 67 |
| improved | | | | | | | | | | | | | | | |
| Fine wrinkles were reduced | | | | | | 1 | | 2 | 3 | 12 | | 24 | 3 | 39 | 42 |
| Skin resilience around eye | | | | 6 | | | | 2 | 1 | 9 | 2 | 14 | 3 | 29 | 29 |
| area was improved | | | | | | | | | | | | | | | |
| Pimples were gone | | | | | | 1 | | | 1 | 1 | 2 | 4 | 3 | 6 | 9 |
| Body was entirely | 1 | 5 | 3 | 1 | 2 | 26 | | 3 | 2 | 8 | 4 | 1 | 12 | 44 | 56 |
| moisturized | | | | | | | | | | | | | | | |
| Color of nails was | | | | | | 3 | 1 | 4 | | 2 | | 1 | 1 | 10 | 11 |
| improved | | | | | | | | | | | | | | | |
| Dry skin was cured | | | | | | 1 | | 2 | | 12 | 1 | 6 | 1 | 21 | 22 |
| Chapped lips were cured | | 4 | 1 | 11 | 3 | 6 | 5 | 8 | 2 | 7 | 1 | 5 | 12 | 41 | 53 |
| Ruddy complexion was | | | | 3 | 1 | 2 | | 8 | 2 | 6 | | 2 | 3 | 21 | 24 |
| improved | | | | | | | | | | | | | | | |
| Stiffness in shoulders was | | 2 | 4 | | 3 | 11 | | 4 | 2 | 4 | 2 | 9 | 11 | 30 | 41 |
| alleviated | | | | | | | | | | | | | | | |
| Risk of hangover was | | | | | | | | 3 | 4 | | 5 | 1 | 9 | 4 | 13 |
| reduced | | | | | | | | | | | | | | | |
| Poor circulation was | | | | 4 | 1 | | | | 3 | 16 | 4 | 12 | 8 | 32 | 40 |
| improved | | | | | | | | | | | | | | | |
| Wounds were healed faster | | | | | | | 1 | | | 2 | | 2 | 1 | 4 | 5 |
| Nails were strengthened | | | | | | | | 3 | | 5 | 1 | 7 | 1 | 15 | 16 |
| Backache was alleviated | | | | 9 | 4 | 5 | 2 | 2 | | 1 | 3 | | 9 | 17 | 26 |
| Memory loss was reduced | | | | 5 | 2 | | | | | 4 | 1 | 2 | 3 | 6 | 9 |
| Blurry vision was cleared | | 8 | 3 | 6 | 2 | 3 | 4 | 4 | 2 | 11 | 1 | 13 | 12 | 44 | 56 |
| Joint pain was alleviated | | 1 | | | 2 | | | | 1 | 3 | 2 | | 5 | 10 | 15 |
| Genital aging was improved | | | | | | | | | 1 | | 2 | | 3 | | 3 |
| Menstrual pain was | | | | | | | | 4 | | 1 | | 2 | | 7 | 7 |
| alleviated | | | | | | | | | | | | | | | |
| Skin of heel was softened | | | | 3 | 3 | 16 | 2 | 4 | 2 | 12 | 2 | 6 | 9 | 38 | 47 |
| Fatigue was reduced | | 1 | | 2 | 2 | 15 | 1 | 6 | 7 | 12 | 2 | 18 | 12 | 55 | 67 |
| Skin looked younger than | | | | | | | | | | 9 | 2 | 13 | 2 | 24 | 26 |
| actual age | | | | | | | | | | | | | | | |
| Constipation was treated | | | 1 | | 1 | 2 | | 7 | 3 | 15 | | 21 | 5 | 45 | 50 |
| Hair loss was reduced | | | | | 1 | 2 | | 3 | 6 | | 10 | 8 | 17 | 13 | 30 |

As can be seen from Table 4, the health food containing hyaluronic acid and dermatan sulfate according to the present invention has the effects of clearing a muddy complexion, reducing spots on the face, improving appearance of makeup, reducing fine wrinkles, improving skin resilience around eye area, clearing pimples, moisturizing the entire body, improving the color of the nails, curing dry skin, curing chapped lips, improving a ruddy complexion, alleviating stiffness in the shoulders, reducing the risk of a hangover or quickly recovering from a hangover, improving poor circulation, healing wounds faster, strengthening the nails, alleviating backache, reducing memory loss, clearing up blurry vision, alleviating joint pain, improving genital aging, alleviating menstrual pain, softening the skin of the heel, recovering from fatigue, rejuvenating the skin, treating constipation, and reducing hair loss. It is apparent from the results that the substance containing hyaluronic acid and dermatan sulfate also functions as an agent for improving constipation and an agent for reducing hair loss.

The question, “Do you think the health food of the present invention has the effect of improving your beauty or health?” was put to the 100 monitors, and their answers are shown in Table 5. It is to be noted that the values in Table 5 represent the number of people.

TABLE 5

| | Effect of | Effect of |
|---|---|---|
| | improving beauty | improving health |
| Yes | 76 | 38 |
| No | 0 | 10 |
| No idea | 24 | 52 |

As can be seen from Table 5, 76 monitors agreed that the health food of the present invention had the effect of improving beauty. This indicates that they realized excellent effects of the health food containing hyaluronic acid and dermatan sulfate according to the present invention.

1 A health food comprising at least hyaluronic acid and dermatan sulfate.

2. An agent for producing beautiful skin and improving constipation, comprising at least hyaluronic acid and dermatan sulfate.

3. The agent for producing beautiful skin and improving constipation according to claim 2, wherein
a mixing ratio between the hyaluronic acid and the dermatan sulfate is 1:0.001 to 200.

4. An agent for producing beautiful skin and reducing hair loss, comprising at least hyaluronic acid and dermatan sulfate.

5. The agent for producing beautiful skin and reducing hair loss according to claim 4, wherein
a mixing ratio between the hyaluronic acid and the dermatan sulfate is 1:0.001 to 200.

6. A health food comprising the agent for producing beautiful skin and improving constipation according to claim 2 or 3.

7. A health food comprising the agent for producing beautiful skin and reducing hair loss according to claim 4 or 5.

8. The health food according to any one of claims 1, 6 and 7, further comprising chondroitin sulfate A and/or chondroitin sulfate C.

9. The health food according to any one of claims 1, 6 and 7, further comprising peptide.
